(19) United States
(12) Patent Application Publication
(10) Pub. No.: US 2007/0128282 A1
(43) Pub. Date: Jun. 7, 2007

(54) Oral osmotic drug delivery system

(76) Inventor: Hasmukh B. Patel, Edison, NJ (US)

Correspondence Address: ARENT FOX PLLC, 1050 CONNECTICUT AVENUE, N.W., SUITE 400, WASHINGTON, DC, 20036, US

(21) Appl. No.: 11/432,670

(22) Filed: May 12, 2006

Related U.S. Application Data
(63) Continuation-in-part of application No. 11/292,305, filed on Dec. 2, 2005.

(51) Int. Cl. A61K 9/24; A61K 31/455

(52) U.S. Cl. 424/473; 514/355

(57) ABSTRACT

The present invention relates, generally, to oral osmotic drug delivery systems, methods of preparing same, and methods of using oral osmotic drug delivery systems to provide controlled delivery of a drug. The oral osmotic drug delivery systems include a drug layer, an osmotic layer, and an outer coating surrounding the drug layer and osmotic layer, where the outer coating includes at least one opening therein that is provided adjacent the drug layer. The composition (% fines), shape, and weight gain of the oral osmotic delivery systems may be modified in order to provide for optimized release of the drug contained therein.

### RELATED APPLICATION DATA

This application is a continuation-in-part of U.S. patent application Ser. No. 11/292,305, which was filed on Dec. 2, 2005, the contents of which are incorporated herein by reference.

### BACKGROUND OF THE INVENTION

1. Field of the Invention

The present invention relates to an oral osmotic delivery system comprising a drug composition and, more particularly, an oral osmotic delivery system and methods of using the oral osmotic delivery system to provide controlled and sustained delivery of a drug. The system makes it possible to obtain an improved release profile for the drug being administered.

2. Description of the Related Art

Pharmaceutical compositions intended for oral administration are typically solid dosage forms (e.g., tablets) or liquid preparations (e.g., solutions, suspensions, or elixirs). The usefulness of an oral formulation, however, requires that the active agent be bioavailable at therapeutic levels over a specific time period of time. For example, for ailments requiring multiple doses of a particular drug, the blood levels of the drug need to be maintained above its minimum effective level and below its minimum toxic level to obtain the desired therapeutic effects, to avoid undesired toxic effects, and to minimize side effects. Thus, methods of controlled drug delivery are desirable to achieve substantially constant blood levels of the active ingredient, as compared to the uncontrolled fluctuations observed when multiple doses of immediate release conventional dosage forms are administered to a patient. Further, methods of controlled drug delivery may not only reduce the frequency of dosing, but may also reduce the severity and frequency of side effects.

Efforts have been made to develop new pharmaceutically viable and therapeutically effective controlled drug delivery systems. In particular, orally administered controlled drug delivery systems have been preferred because of the ease of administration via the oral route as well as the ease and economy of manufacturing oral dosage forms such as tablets and capsules. Various oral controlled drug delivery systems based on different release mechanisms have been developed, for example, dissolution controlled systems, diffusion controlled systems, ion-exchange resins, osmotically controlled systems, erodible matrix systems, pH-independent formulations and swelling controlled systems.

Osmotic dosage forms utilize osmotic pressure to absorb fluid through a semi-permeable wall, which permits free diffusion of fluid but not the drug or the osmotic agent. The drug is pushed out of a hole or opening in the dosage form. A significant advantage to osmotic systems is that their operation is pH-independent. A review of osmotic delivery forms is provided in Santus et al., Journal of Controlled Release 35, 1-21 (1995), the disclosure of which is hereby incorporated by reference in its entirety. In addition, U.S. Pat. Nos. 3,845,770; 3,916,899; 3,995,631; 4,008,719; 4,111,202; 4,160,020; 4,327,725; 4,519,801; 4,578,075; 4,681,583; 5,019,397; and 5,156,850 are each related to osmotic dosage forms, and their disclosures are also hereby incorporated by reference in their entirety.

U.S. Pat. Nos. 4,816,263; 4,950,486; 4,946,687; and 5,030,456 relate to methods for treating a cardiovascular disease by providing an oral dosage form that can deliver isradipine over a prolonged period of time at a controlled rate and in a constant dose per unit time.

Sastry et al. disclose that various factors, such as orifice size, coating thickness, amount and nature of polymeric excipients, and amount of osmotic agent influence the drug release from Gastrointestinal Therapeutic Systems (GITS). In particular, the in vitro atenolol release rate of atenolol GITS coated with CA pseudolatex is influenced by orifice size, % coating weight gain, and amount of Carbopol 934P. Furthermore, drug release under constrained conditions is influenced by the following factors, in decreasing order of importance: % coating weight gain>Carbopol 934P>Polyox N80>Carbopol 974P>Polyox 303>amount of sodium chloride>orifice size. Sastry et al., Pharm Acta Helv. 73(2):105-12 (1998).

Although osmotic dosage forms have been disclosed, there remains a need in the art for an oral osmotic delivery system with improved performance. In particular, an oral osmotic delivery system that can provide reliable and effective controlled release of a drug after administration to a subject, especially an improved 16-hour cumulative dissolution of drug.

The present inventors have found that a number of factors can be controlled in an oral osmotic delivery system to achieve an improved 16-hour cumulative dissolution of drug. For example, the make-up of the drug layer, the shape of the tablet, and the tablet weight gain can each be controlled for optimum performance. These attributes are neither disclosed nor suggested by the prior art.

An oral osmotic delivery system for controlled release of isradipine is currently being marketed by Reliant Pharmaceuticals, Inc. under the trade name DYNACIRC® CR. Prior batches of DYNACIRC® CR providing a 5 mg label claim of isradipine had an upper limit of about 22% fines (defined below), a maximum release rate of about 8.4%/hr, and achieved a maximum average 16 hour cumulative dissolution (hereinafter referred to as “Q value”) of approximately 90%, when measured according to the following conditions: USP dissolution test <711>, Apparatus 2, 50 rpm, 500 ml (for 5 mg tablets) or 1000 ml (for 10 mg tablets) of 0.2% N,N-dimethyl dodecylamine N-oxide (LDAO) or equivalent solvent, temperature 37° C.±0.5° C. Prior batches of DYNACIRC® CR providing a 10 mg label claim of isradipine had an upper limit of about 26% fines, a maximum release rate of about 8.8%/hr, and achieved a maximum Q value of approximately 102%. Certain embodiments of the present invention represent an improvement to this oral osmotic delivery system.

U.S. Pat. Nos. 4,612,008 and 5,232,705 disclose conventional oral osmotic delivery systems, in which the drug layer of the system is prepared using particles of drug and/or polymer that pass through a 40 mesh screen (i.e., 400 microns or less). A wet granulation of drug, polymer and granulating agents is passed through a 16 mesh screen (i.e., 1190 microns or less). See Example 3 of the '008 patent and Example 4 of the '705 patent.

U.S. Pat. No. 6,096,339 discloses that a more constant drug release pattern can be achieved in an oral osmotic delivery system by using in the drug layer particles of a drug having a size between 1 to 150 microns and particles of a polymer having a size between 1 to 250 microns. These drug and polymer particles are combined with granulating agents to achieve a blend of undisclosed granule size. The patent discloses that the wet granulate may be forced through a predetermined screen size, although no examples are provided.

U.S. Pat. No. 6,514,530 is directed to an oral osmotic drug delivery system which changes from a non-rounded to a rounded shape to improve delivery of a drug. The shape change is effectuated by the addition of a plasticizer to the outer coat of the system, allowing for flexibility of the dosage form.

Accordingly, none of the above-mentioned approaches provides oral osmotic drug delivery systems, methods for preparing oral osmotic drug delivery systems, and methods for treating diseases and/or conditions using the oral osmotic drug delivery systems that are described herein with respect to the present invention.

There is clearly a great need in the art for oral osmotic drug delivery systems providing improved release profiles. Methods of treating diseases and/or other medical conditions using the oral osmotic drug delivery systems are also needed. There is an especially great need in the art for oral osmotic drug delivery systems useful for administering dihydropyridines, such as israpidine.

### SUMMARY OF THE INVENTION

The present invention relates to an oral osmotic delivery system comprising a drug composition and, more particularly, an oral osmotic delivery system and methods of using the oral osmotic delivery system that provide controlled and sustained delivery of a drug, especially an improved 16-hour cumulative dissolution of drug. There is an unmet need in the art for such compositions and methods.

The present invention meets the unmet needs of the art, as well as others, by providing a 16-hour cumulative release of drug (Q) of over 85%, preferably at least 90%, more preferably at least 92.5%, still more preferably at least 95%, and most preferably at least 97.5%. These Q values may be achieved in a variety of ways, as described herein. Notably, these Q values may be achieved by adjusting the amount of fines in the drug layer, by adjusting the shape of the dosage form, or by adjusting the thickness of the outer coating, or by adjusting a combination of these elements. In all embodiments described herein, the Q value is measured according to the following dissolution conditions: USP dissolution test <711>, Apparatus 2, 50 rpm, 0.2% N,N-dimethyl dodecylamine N-oxide (LDAO) or equivalent solvent, temperature 37° C.±0.5° C. For tablets of 5 to 10 mg drug dose set forth in the label claim, 100 ml of solvent per mg of drug is used. For tablets of less than 5 mg drug dose set forth in the label claim, two tablets are used in the dissolution test, and 100 ml of solvent per the total mg weight of drug in the two tablets is used. For tablets of more than 10 mg drug dose set forth in the label claim, 1000 ml of solvent is used.

In some embodiments, the present invention is directed to an osmotic drug delivery system for delivering a drug to a subject, including a drug layer having a drug and one or more non-drug ingredients including a hydrophilic polymer having a molecular weight of from about 40,000 to about 750,000, where the drug and non-drug ingredients are combined to form particles with more than 28% fines; an osmotic layer including a hydrophilic polymer having a molecular weight of from about 1,000,000 to about 15,000,000; and a semi-permeable outer wall that substantially surrounds the drug layer and the osmotic layer, and defines at least one opening in communication with the drug layer. The osmotic drug delivery system provides a 16-hour cumulative release of drug of at least 85%. In one variation, the drug layer is formed by compressing granules comprising from about 30 to 60% fines. In other embodiments, the present invention is directed to a method of delivering a drug to a subject in need thereof, including administering to the subject the osmotic drug delivery system.

In other embodiments, the present invention is directed to an oral osmotic tablet, including an upper surface having a radius of curvature and defining at least one opening, a lower surface, and a cylindrical sidewall region, having a sidewall radius, the sidewall region disposed between the upper surface and the lower surface, wherein a drug layer, including a drug and one or more non-drug ingredients including a hydrophilic polymer having a molecular weight of from about 40,000 to about 750,000, is disposed toward the upper surface and in communication with the at least one opening, and an osmotic layer, comprising a hydrophilic polymer having a molecular weight of from about 1,000,000 to about 15,000,000, is disposed toward the lower surface, and wherein the radius of curvature of the upper surface and the sidewall radius are in a ratio of greater than 1 to less than 2.7, and wherein said oral osmotic tablet provides a 16-hour cumulative release of drug of at least 85%. According to one variation of this embodiment, the radius of curvature of the upper surface and the sidewall radius are in a ratio of from about 1.05 to about 2.4. According to certain variations of this embodiment, the drug layer may include 2.5 to 20 mg of a dihydropyridine, such as isradipine. In other embodiments, the present invention is directed to a method of delivering a drug to a subject in need thereof, including administering to the subject the oral osmotic tablet.

According to further embodiments, the present invention is directed to an oral osmotic tablet including a drug layer with 2.5 to less than 8 mg of israpidine; an osmotic layer having at least one hydrophilic polymer having a molecular weight of from about 1,000,000 to about 15,000,000; and a semi-permeable outer wall, wherein the outer wall substantially surrounds the drug layer and the osmotic layer, and defines at least one opening to the drug layer, wherein said outer wall has a thickness selected such that the tablet has a weight gain of about 12-20 mg. The oral osmotic tablet provides a 16-hour cumulative release of drug of at least 85%. According to one variation, the oral osmotic tablet has a weight gain of about 15-17 mg. Also provided is a method of delivering israpidine to a subject in need thereof, by administering to the subject such an oral osmotic tablet.

In still other embodiments, the present invention is directed to an oral osmotic tablet including a drug layer comprising an 8 to 20 mg dose of israpidine; an osmotic layer having at least one hydrophilic polymer having a molecular weight of from about 1,000,000 to about 15,000,000; and a semi-permeable outer wall, wherein the outer wall substantially surrounds the drug layer and the osmotic layer, and defines at least one opening to the drug layer, wherein said outer wall has a thickness selected such that the tablet has a weight gain of about 18-26 mg. The oral osmotic tablet provides a 16-hour cumulative release of drug of at least 85%. According to one variation, the oral osmotic tablet has a weight gain of about 21-23 mg. The present invention also provides a method of delivering israpidine to a subject in need thereof, including administering to the subject the oral osmotic tablet.

In still further embodiments, the present invention is directed to an oral osmotic tablet including a drug layer comprising a 2.5 to 20 mg dose of isradipine; an osmotic layer having at least one hydrophilic polymer having a molecular weight of from about 1,000,000 to about 15,000,000; and a semi-permeable outer wall, wherein the outer wall substantially surrounds the drug layer and the osmotic layer, and defines at least one opening to the drug layer, wherein said outer wall has a thickness selected such that the tablet has a weight gain of about 12-26 mg. The oral osmotic tablet provides a 16-hour cumulative release of drug of at least 85%. According to one variation, the oral osmotic tablet has a weight gain of about 15-23 mg. According to another variation, the oral osmotic tablet has a weight gain of about 17-21 mg. The present invention also provides a method of delivering israpidine to a subject in need thereof, including administering to the subject the oral osmotic tablet.

In additional embodiments, the present invention is directed to an osmotic drug delivery system for delivering a drug to a subject including a drug layer including a drug and one or more non-drug ingredients including a hydrophilic polymer having a molecular weight of from about 40,000 to about 750,000; an osmotic layer comprising a hydrophilic polymer having a molecular weight of from about 1,000,000 to about 15,000,000; and a semi-permeable outer wall, wherein the outer wall substantially surrounds the drug layer and the osmotic layer, and defines at least one opening in communication with the drug layer. The osmotic drug delivery system provides a 16-hour cumulative release of drug of greater than 85%. In still further embodiments, the present invention is directed to a method of delivering a drug to a subject in need thereof, by administering to the subject the osmotic drug delivery system.

Other novel features and advantages of the present invention will become apparent to those skilled in the art upon examination of the following or upon learning by practice of the invention.

### BRIEF DESCRIPTION OF THE DRAWINGS

FIG. 1 is a diagram that shows the relative dimensions of a preferred oral osmotic tablet;

FIG. 2 is a graph that shows the effect of % Fines on 16-hour cumulative drug dissolution for an exemplary 5 mg israpidine tablet;

FIG. 3 is a graph that shows the effect of weight gain on 16-hour cumulative drug dissolution for an exemplary 10 mg israpidine tablet; and

FIG. 4 shows three different exemplary embodiments of the shape of an oral osmotic drug delivery system of the present invention.

### DETAILED DESCRIPTION OF THE INVENTION

The present invention relates to an oral osmotic delivery system comprising a drug composition and, more particularly, an oral osmotic delivery system and methods of using the oral osmotic delivery system to provide controlled and sustained delivery of a drug to a subject. The oral osmotic drug delivery systems of the present invention may be beneficially configured to permit once-daily dosing schedules of a variety of drugs.

Generally, oral osmotic drug delivery devices can be manufactured in a number of forms, including capsules, caplets, and tablets. The tablet form is preferred in accordance with the present invention.

A preferred oral osmotic drug delivery device includes a drug layer comprising a drug, and optionally comprising one or more non-drug ingredients, such as hydrophilic polymers, osmagents, binding agents, antioxidants, solvents, etc. The oral osmotic drug delivery device further includes an osmotic layer comprising a hydrophilic polymer that swells on contact with fluid present outside the exterior of the dosage form, and optionally comprising one or more additional ingredients.

The oral osmotic drug delivery device further includes an outer wall that surrounds the drug layer and the osmotic layer. The outer wall is formed at least in part from a semi-permeable composition, i.e., one that is permeable to the passage of an exterior fluid present in the environment of use, and is substantially impermeable to the passage of the drug and other non-drug ingredients present within the outer wall. The outer wall is substantially inert, and it maintains its physical and chemical integrity during the dispensing life of the drug from the dosage form.

The outer wall of the oral osmotic drug delivery device includes at least one exit means that connects the drug layer with the environment surrounding the exterior of the dosage form. The exit means is preferably a hole drilled in the outer wall of the tablet.

In operation, the fluid found in the environment surrounding the oral osmotic drug delivery device permeates through the outer wall and is absorbed by the osmotic layer, causing the osmotic layer to expand. Because the outer wall substantially maintains its physical shape despite the expansion of the osmotic layer, this causes the drug layer to be compressed against the outer wall. The drug layer is therefore pushed out of the hole in the outer wall by the expansion of the osmotic layer. The drug contained in the oral osmotic delivery device is therefore released into the environment.

It has been discovered in accordance with the present invention that various modifications to the oral osmotic tablet dosage form described above provide advantageous improvements in the release characteristics of the dosage form, thereby providing an improved 16-hour cumulative dissolution (Q value) of the drug ingredient(s) contained therein. These modifications are described in further detail below with respect to the preferred embodiments of the present invention.

1. Drug Layer

According to the present invention, the oral osmotic delivery system includes a drug layer that comprises a drug, and optionally one or more non-drug ingredients.

Any drug known in the art, typically provided through oral administration, may be used according to the present invention. Agents that can be delivered according to this invention are those that are compatible with the components of the oral osmotic drug delivery system.

According to the present invention, the amount of drug and non-drug ingredients provided in the oral osmotic delivery system is not critical. Generally, the delivery system can house from about 0.05 ng to 500 mg or more of drug, carriers, fillers, excipients, etc., with individual tablets being configured to contain, for example, 25 ng, 1 mg, 5 mg, 10 mg, 50 mg, 125 mg, 250 mg, 500 mg, and the like.

Particularly preferred oral osmotic tablets in accordance with the present invention contain a dose of a dihydropyridine, such as amlodipine, felodipine, israpidine, lacidipine, lercanidipine, nicardipine, nifedipine, nimodipine, nisoldipine, and nitrendipine. The dose of dihydropyridine may be from about 1 mg to about 150 mg daily. More specifically, for example, when the dihydropyridine is felodipine, the preferred daily dose is about 2.5-20 mg daily. When the dihydropyridine is nifedipine, the preferred daily dose is about 20-120 mg daily. When the dihydropyridine is amlodipine, the preferred daily dose is about 5-10 mg daily. When the dihydropyridine is nicardipine, the preferred daily dose is about 20-120 mg daily. A particularly preferred dihydropyridine is israpidine, which may be administered in a preferred daily dose of 2.5-20 mg daily. Most preferably, a 5 or 10 mg label claim dose of israpidine is administered once daily using the oral osmotic drug delivery system of the present invention. As will be understood by those of skill in the art, the amount of the particular drug agent employed in the delivery device is that amount necessary to deliver a therapeutically effective amount of the drug to achieve the desired therapeutic result. In practice, this will vary depending on the particular agent, the severity of the condition, and the desired effect, as well as the desired rate and duration of release.

It is known from U.S. Pat. No. 6,514,530 that some amount of drug may remain in the oral osmotic delivery system after the dissolution is complete. Generally, this amount is from about 7 to about 25%, as set forth in DYNACIRC® CR NDA #20-336. Accordingly, oral osmotic delivery system dosage forms generally contain an amount of drug in excess of the label claim, e.g., from about 7-25% excess. Therefore, the present invention includes embodiments whereby from about 7-25% excess of the label claim is included in the dosage form. For example, an israpidine oral osmotic tablet with a 5 mg label claim may contain about 10-20% excess drug. An israpidine oral osmotic tablet with a 10 mg label claim may contain about 5-15% excess drug. In other preferred embodiments of the present invention, there is no excess drug provided in the tablet, i.e., the tablet contains not more than 100% of the drug label claim.

The drug layer of the oral osmotic delivery system according to the present invention may further include a hydrophilic polymer. When provided, the hydrophilic polymer is present in an amount ranging from about 20-99.5 weight %, more preferably from about 30-90 weight %, based on the weight of the drug layer. Preferred hydrophilic polymers in the drug layer are those having a molecular weight of about 40,000 to about 750,000. Hydrophilic polymers that may be used in the drug layer of the present invention include any polymers that interact with water and/or aqueous biological fluids and swell or expand to a high degree, typically exhibiting a 2-50 fold volume increase. In some embodiments according to the present invention, the hydrophilic polymer of the drug layer comprises a poly(alkylene oxide), including, but not limited to, poly(ethylene oxide), poly(methylene oxide), poly(butylene oxide) and poly(hexylene oxide), preferably at 100,000 to 750,000 molecular weight. In other embodiments according to the present invention, the hydrophilic polymer of the drug layer comprises a poly(carboxymethylcellulose), including, but not limited to, poly(alkali carboxymethylcellulose) such as poly(sodium carboxymethylcellulose), poly(potassium carboxymethylcellulose) and poly(lithium carboxymethylcellulose), preferably at 40,000 to 400,000 molecular weight. In some embodiments according to the present invention the hydrophilic polymer comprises (poly)hydroxypropylmethyl cellulose, (poly)hydroxypropyl cellulose, or (poly)vinyl alcohol. In some embodiments according to the present invention the hydrophilic polymer comprises poly(ethylene oxide) with a molecular weight of from about 100,000 to about 300,000. In a preferred embodiment, the poly(ethylene oxide) has a molecular weight of about 200,000.

According to another alternative embodiment, an osmagent may optionally be provided in the drug layer of the oral osmotic delivery system, when the drug being administered is not itself osmotically active, or to further increase the osmotic activity. These osmagents are soluble in the fluid that enters the device, and exhibit an osmotic pressure gradient across the semi-permeable wall against the exterior fluid. When provided, the osmagent is present in an amount ranging from about 140 weight %, based on the weight of the drug layer. Osmotically-effective osmagents useful for the present purpose include magnesium sulfate, magnesium chloride, sodium chloride, lithium chloride, potassium sulfate, sodium carbonate, sodium sulfite, lithium sulfate, potassium chloride, sodium sulfate, d-mannitol, urea, sorbitol, inositol, raffinose, sucrose, glycose, hydrophilic polymers such as cellulose polymers, mixtures thereof, and the like. The osmagent is usually present in an excess amount, and it can be in any physical form, such as particle, powder, granule, and the like. The osmotic pressure in atmospheres of the osmagents suitable for the invention will be greater than zero and generally up to about 500 atm, or higher.

Additional non-drug ingredients may optionally be included in the drug layer of the oral osmotic delivery system of the present invention. Such non-drug ingredients may include binding agents (e.g., hydroxypropyl methylcellulose) in an amount of from about 0.5-10 weight %, antioxidants (e.g., butylated hydroxy toluene) in an amount of from about 0.5-10 weight %, lubricants in an amount of from about 0.01-3.0 weight %, and colorants in an amount of from about 0-3 weight %. All weight percentages are based on the weight of the drug layer. Solvents, such as alcohols, may also be provided in an amount suitable for aiding in preparing the drug layer, but any solvents are preferably evaporated out of the finished drug layer.

Preferably, the drug layer will include about 0.1-50 weight % drug, about 30-99.5 weight % hydrophilic polymer, and about 0-15 weight % of other optional non-drug ingredients. In one embodiment according to the present invention, the drug layer of the oral osmotic delivery system comprises from about 2-30 weight % isradipine and from about 30-95 weight % of a polyethylene oxide. A more preferred drug layer in accordance with the present invention comprises 80-90 weight % polyethylene oxide, 3-6 weight % israpidine, 5-14 weight % hydroxypropyl methylcellulose, and up to 0.2 weight % butylated hydroxytoluene. A particularly preferred drug layer in accordance with the present invention comprises 85 weight % polyethylene oxide, 5 weight % israpidine, 10 weight % hydroxypropyl methylcellulose, 0.25 weight % colloidal silicon dioxide, and 0.05 weight % butylated hydroxytoluene. All weight percentages are based on the weight of the drug layer.

Any method known in the art may be used to combine the drug and non-drug ingredients together in order to produce the drug layer of the present invention. These methods include, but are not limited to, granulation, spray drying, sieving, lyophilization, crushing, grinding, jet milling, micronizing, and chopping. The size of the particles containing the drug and non-drug ingredients can be ascertained by screening, using, for example, a grizzly screen, a flat screen, a vibrating screen, a revolving screen, a shaking screen, an oscillating screen or a reciprocating screen. The processes and equipment for preparing drug and carrier particles are disclosed in Pharmaceutical Sciences, Remington, 17th Ed., pp. 1585-1594 (1985); Chemical Engineers Handbook, Perry, 6th Ed., pp. 21-13 to 21-19 (1984); Journal of Pharmaceutical Sciences, Parrot, Vol. 61, No. 6, pp. 813-829 (1974); and Chemical Engineer, Hixon, pp. 94-103 (1990), the disclosures of which are hereby incorporated by reference in their entirety.

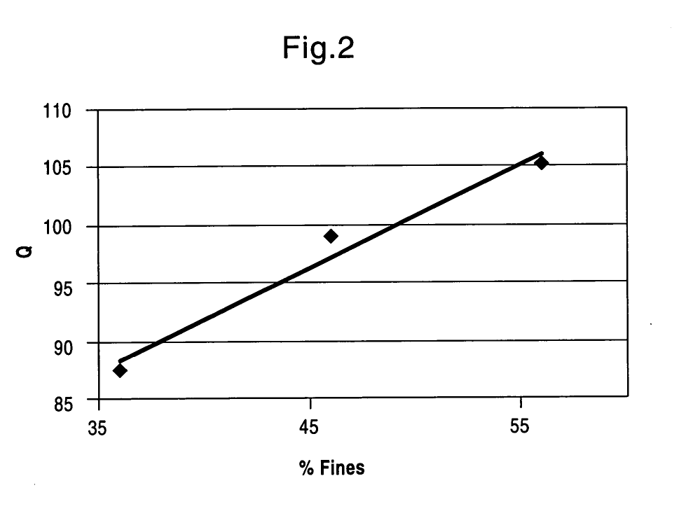

It has been determined in accordance with the present invention that there is a relationship between % fines in the particles that are compressed to form the drug layer and 16-hour cumulative dissolution (Q), such that as % fines increases, Q also increases. FIG. 2 shows this trend. “Fines” are herein defined as those particles comprising drug and non-drug ingredients that are capable of passing through a US #120 mesh sieve; or particles of equal to or less than 125 microns in size. The % fines in the drug layer is at least 23%, and is more preferably over 28%. Most preferably, the % fines in the drug layer is about 30-60%. Preferably, the granules in the drug layer are produced by combining the drug and non-drug ingredients and thereafter screening the mixture to ensure that the granules have the % fines expressed above.

2. Osmotic Layer

According to the present invention, the oral osmotic delivery system further includes an osmotic layer. The osmotic layer comprises one or more hydrophilic polymers. Preferably, these hydrophilic polymers have a molecular weight of about 1,000,000 to about 15,000,000. Hydrophilic polymers that may be used in the osmotic layer of the present invention include any polymers that interact with water and aqueous biological fluids and swell or expand to a high degree, typically exhibiting a 2-50 fold volume increase. For example, hydrophilic polymers that may be used in the osmotic layer of the present invention may include the same types of polymers indicated above for the drug layer. Preferably, the hydrophilic polymer is selected from a poly(alkylene oxide), including, but not limited to, poly(ethylene oxide), and poly (alkalicarboxymethylcellulose).

The osmotic layer comprises an hydrophilic polymer having a greater number molecular weight than the hydrophilic polymer present in the drug layer. The osmotic layer preferably comprises a member selected from the group consisting of polyalkylene oxide of 1,000,000 to 10,000,000 weight-average molecular weight. Representative of such polyalkylene oxides are polyethylene oxide of 1 million molecular weight, polyethylene oxide of 2 million molecular weight, polypropylene oxide of 4 million molecular weight, polyethylene oxide of 5 million molecular weight, and polyethylene oxide of 7.5 million molecular weight. The hydrophilic polymer may also be selected from carboxyalkyl celluloses of 200,000 to 3,250,000 weight-average molecular weight. Representative of such carboxyalkyl celluloses are lithium carboxymethyl cellulose, potassium carboxymethyl cellulose of 200,000 molecular weight, sodium carboxymethyl cellulose of 200,000 molecular weight, sodium carboxymethyl cellulose of 1,250,000 molecular weight, potassium carboxymethyl cellulose of 1,500,000 molecular weight, sodium carboxyethyl cellulose of 2,250,000 molecular weight, and sodium carboxymethyl cellulose of 3,250,000 molecular weight. The amount of hydrophilic polymer present in the osmotic layer is preferably from about 20-100 weight %, and is more preferably from about 40-75 weight %, based on the weight of the osmotic layer.

The osmotic layer may optionally comprise from about 5-50 weight % of an osmagent, more preferably 10-40 weight %, based on the weight of the osmotic layer. The osmagent exhibits an osmotic pressure gradient across the outer wall, and imbibes fluid into the oral osmotic dosage form that aids in the expansion of the osmopolymer, thereby promoting the displacement of the drug layer from the dosage form. Representative osmagents include those selected from the group consisting of sodium chloride, potassium chloride, magnesium sulfate, magnesium chloride, lithium sulfate, lithium chloride, lithium phosphate, sodium phosphate, potassium sulfate, potassium sulfite, sodium sulfate, potassium nitrate, and potassium phosphate, and the like. Other osmotically-effective compounds are described, for example, in U.S. Pat. Nos. 4,177,256 and 4,449,983.

The osmotic layer may also optionally comprise a binder, such as hydroxyalkyl cellulose, hydroxypropyl celluloses, hydroxypropyl alkylcelluloses and polyvinyls. The hydroxypropyl alkylcelluloses possess a 9,000 to 400,000 number-average molecular weight, and may be selected from the group consisting of hydroxypropyl methylcellulose, hydroxypropyl ethylcellulose, hydroxypropyl butylcellulose, hydroxypropyl pentylcellulose, and hydroxypropyl hexylcellulose. The polyvinyls of 1,200 to 360,000 viscosity-average molecular weight comprising, a member selected from the group consisting of polyvinylpyrrolidone, polyvinylcarbazole, polyvinylpyridine, polyvinyloxazole, polyvinylmethyloxazolidone, polyvinylbutyrol, polyvinylacetate, polyvinylalcohol, copolymers of polyvinylprrolidone and vinyl acetate, copolymers of polyvinylpyrrolidone and vinyl alcohol, copolymers of polyvinylpyrrolidone and vinyl chloride, copolymers of polyvinylpyrrolidone and vinyl fluoride, copolymers of polyvinylpyrrolidone and vinyl butyrate, copolymers of polyvinylpyrrolidone and vinyl laurate, and copolymers of polyvinylpyrrolidone and vinyl stearate. The amount of binder in the osmotic layer is preferably from about 0.5-15 weight %, based on the weight of the osmotic layer.

The osmotic layer may also optionally comprise from about 0-2.75 weight % of a colorant, preferably a non-toxic colorant, and more preferably a colorant selected from Food and Drug Administration colorants such as FD&C No. 1 blue, ferric oxide, and the colorants disclosed above. The osmotic layer may also optionally comprise from about 0.05-3.75 weight % of a lubricant, such as sodium stearate, potassium stearate, magnesium stearate, stearic acid, calcium stearate, calcium palmitate, potassium oleate. All weight percentages are based on the weight of the osmotic layer.

According to one embodiment, the osmotic layer includes about 20-95 weight % hydrophilic polymer, about 5-50 weight % osmagent, about 1-15 weight % binder, and about 0-6 weight % of other optional ingredients. All weight percentages are based on the weight of the osmotic layer.

3. Optional Additional Layer(s)

According to the present invention, the oral osmotic delivery system may optionally include additional drug layers, osmotic layers, and/or internal piston layers. When provided, the additional drug layer(s) may be included in order to achieve a particular release profile of the drug being administered, for example, by including more than one layer of the same drug, where the drug layers have different formulations (e.g., each drug layer includes a different hydrophilic polymer). Such additional drug layers may also be used to provide for co-administration of additional compounds useful in treating a particular disease or condition. When additional osmotic layer(s) are provided, these may be useful for achieving a particular release profile. This may be accomplished, for example, by utilizing osmotic layer(s) that have different swelling characteristics. The optional additional piston layer may be provided to assist in displacing the drug layer from the dosage form.

4. Tablet Configuration

The oral osmotic drug delivery system of the present invention is preferably provided in the form of a tablet. The particularly preferred tablet shape in accordance with the present invention will be described below.

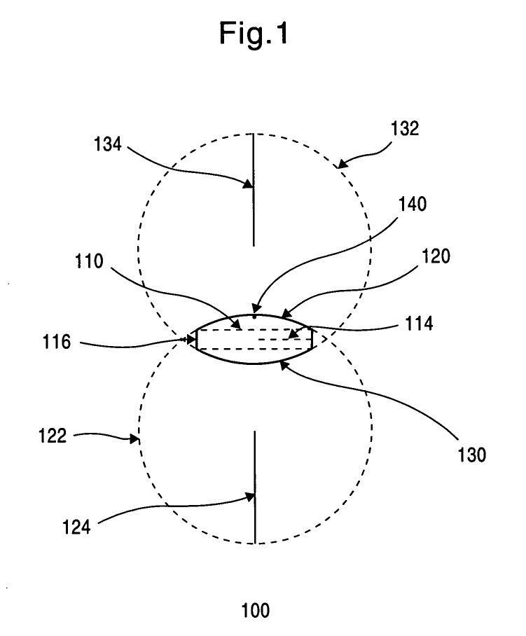

With reference to FIG. 1, the tablet 100 (shown in solid lines) is configured such that it has a sidewall or band region 110 (shown in dotted lines) provided between upper and lower surfaces 120, 130. The sidewall or band region 110 is cylindrical in shape, with the sidewall having a height 116 that is selected so as to provide ease of oral administration (i.e., swallowing), and wherein the radius of the cylindrical sidewall region 114 defines the radius of the tablet. The upper and lower surfaces 120, 130 of the tablet 100 may each be characterized as portions of theoretical spheres 122, 132 having radii 124, 134 defining a curvature. The curvature of the theoretical spheres 122, 132 intersects the top and bottom edges, respectively, of the cylindrical sidewall 116. The upper surface of the tablet 120 includes a drilled orifice 140 in the outer wall, and the drug layer is provided toward the upper surface 120 and in communication with the orifice 140. The osmotic layer is provided toward the lower surface 130 of the tablet. The radius 134 of theoretical sphere 132 (corresponding to the lower surface of the tablet) may or may not be the same as the radius 124 of theoretical sphere 122 (corresponding to the upper surface).

It has been found in accordance with the present invention that when the radius of curvature of the upper surface of the tablet decreases (i.e., the degree of concavity of the mold or punch used to form the upper surface of the tablet increases), the 16-hour cumulative dissolution of the drug improves. This may be expressed as a ratio of the radius of curvature of said upper surface to the radius of the tablet (as defined by the radius of its cylindrical sidewall region). Preferred ratios of the radius of curvature to the tablet radius are greater than 1, and less than 3. More preferred ratios of the radius of curvature to the tablet radius are greater than 1, and less than 2.8.

In one preferred embodiment, a 5 mg israpidine oral osmotic tablet has an upper surface with a radius of curvature of greater than 4 mm and less than 9.5 mm when the tablet has a radius of about 4 mm. Preferably, the radius of curvature of the upper surface of the tablet is from about 4.5 mm to about 8 mm. Most preferably, the radius of curvature of the upper surface of the tablet is from about 5.93 mm to about 7.19 mm.

The tablets of the present invention containing a drug in an amount of from 2 to less than 8 mg may have a ratio of radius of upper surface curvature:tablet radius from greater than 1 to less than 2.4. Preferably, the tablets have a ratio of radius of upper surface curvature:tablet radius from about 1.125 to about 2. Most preferably, the tablets have a ratio of radius of upper surface curvature:tablet radius from about 1.5 to about 1.8.

In another preferred embodiment, a 10 mg israpidine oral osmotic tablet has an upper surface with a radius of curvature of greater than 4.8 mm and less than 12.8 mm when the tablet has a radius of about 4.8 mm. Preferably, the radius of curvature of the upper surface is from about 5 mm to about 11.5 mm. Most preferably, the radius of curvature of the upper surface is from about 7.1 mm to about 9.6 mm.

The tablets of the present invention containing a drug in an amount of from 8 to 20 mg may have a ratio of radius of upper surface curvature:tablet radius from greater than 1 to less than 2.7. Preferably, the tablets have a ratio of radius of upper surface curvature:tablet radius from about 1.05 to about 2.4. Most preferably, the tablets have a ratio of radius of upper surface curvature:tablet radius from about 1.5 to about 2.

The radius of curvature of the lower surface of the tablet may be the same as the radius of curvature of the upper surface of the tablet, or it may be different.

5. Outer Coating

According to the present invention, the oral osmotic delivery system comprises an outer coating or wall that substantially surrounds the drug layer and the osmotic layer. The outer coating or wall may serve one or more functions: it may permit the passage of water into the tablet in a continuous manner to provide drug delivery at a steady rate; it may control the rate at which water will pass into the tablet; and/or it may prevent non-volatile materials residing in the tablet from passing through the outer wall. In preferred embodiments, the outer wall is formed at least in part of a semi-permeable material, that is, it is permeable to the inward passage of water from the environment of use, e.g., gastrointestinal fluid, but is substantially impermeable to the outward passage of the materials comprising the drug layer and osmotic layer.

Because the outer wall functions as a pump housing, it must be capable of maintaining its physical and chemical integrity (not distend or disintegrate substantially) during the life of the delivery system. Accordingly, the outer wall is usually between about 0.1-2000 microns thick. Materials from which osmosis and reverse osmosis membranes are made may be used to make the outer wall. Examples of such materials are cellulose acetate, plasticized cellulose triacetate, agar acetate, amylose triacetate, β-glucan acetate, acetaldehyde dimethyl acetate, cellulose acetate ethyl carbamate, cellulose acetate phthalate, cellulose acetate succinate, cellulose acetate dimethaminoacetate, cellulose acetate ethyl carbonate, cellulose acetate chloroacetate, cellulose acetate ethyl oxalate, cellulose acetate methyl sulfonate, cellulose ethers, poly(vinyl methyl) ether copolymers, cellulose acetate octoate, methyl cellulose, polyurethanes, hydrolyzed polyvinylacetate and hydroxylated ethylenevinylacetate copolymer.

The outer wall of the oral osmotic dosage form can be applied, for example, by using an air suspension procedure in which a pressed tablet is suspended and tumbled in a current of air and wall-forming composition until an outer wall is applied onto the tablet. The air suspension procedure is described in greater detail in U.S. Pat. No. 2,799,241; J. Am. Pharm. Assoc., Vol. 48, pp. 451-459 (1959); and J. Am. Pharm. Assoc., Vol. 49, pp. 82-84 (1960). The outer wall can also be formed using a Wurster® air suspension coater and an organic solvent. An Aeromatic® air suspension coater can also be used for applying the outer wall. Other techniques for applying the outer wall may include pan coating, in which the outer wall-forming compositions are deposited by successive spraying of the composition accompanied by tumbling in a rotating pan. The outer wall or coating may also be applied by molding, spraying or dipping the tablet into an outer wall-forming material. Other manufacturing procedures are described in Modern Plastics Encyclopedia, Vol. 46, pages 62 to 70, 1969; and in Pharmaceutical Sciences, by Remington, Fourteenth Edition, pages 1626 to 1678, 1970, published by Mack Publishing Company, Easton, Pa. Finally, after the outer wall of the oral osmotic tablet has been applied, it is laser or mechanically drilled to form at least one passageway in communication with the drug layer, and it is then dried to remove the solvent.

Exemplary solvents suitable for use in applying the outer wall include inert inorganic and organic solvents that do not adversely harm the materials and the final wall. The solvents may include aqueous solvents, alcohols, ketones, esters, ethers, aliphatic hydrocarbons, halogenated solvents, cycloaliphatics, aromatics, heterocyclic solvents, and mixtures thereof. Typical solvents include acetone, diacetone alcohol, methanol, ethanol, isopropyl alcohol, butyl alcohol, methyl acetate, ethyl acetate, isopropyl acetate, n-butyl acetate, methyl isobutyl ketone, methylpropyl ketone, n-hexane, n-heptane, ethylene glycol monoethyl ether, ethylene glycol monoethyl acetate, methylene dichloride, ethylene dichloride, propylene dichloride, acetone and water, acetone and methanol, acetone and ethyl alcohol, methylene dichloride and methanol, ethylene dichloride and methanol, and the like.

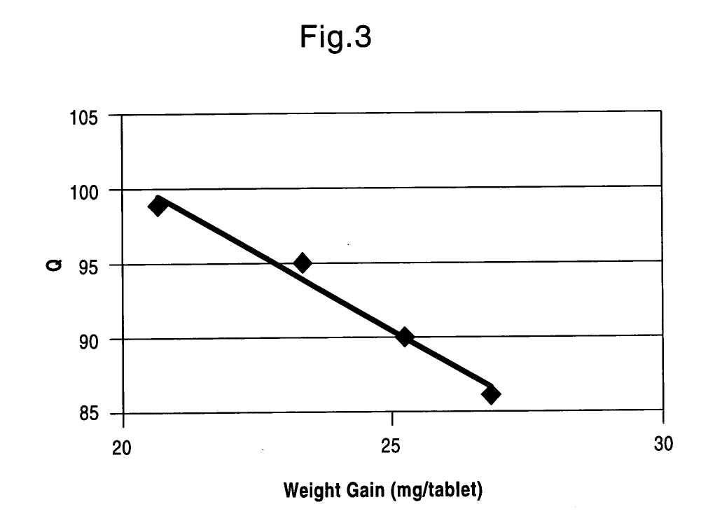

The thickness of the outer coating or wall in accordance with the present invention is formed to a desired level of tablet weight gain. The rate at which external fluid permeates the outer coating or wall is inversely proportional to the thickness of the outer coating (expressed in terms of tablet weight gain) of the oral osmotic delivery system. The present invention further relates to the discovery that the weight gain of the delivery system affects the 16 hour cumulative dissolution (Q value) of the drug layer, where increased weight gain results in decreased cumulative dissolution. For example, in accordance with the present invention, it has been found that for a 2.5 mg to less than 8 mg dose of israpidine provided in an oral osmotic delivery system, an outer wall thickness that permits a weight gain of 12-20 mg is preferable, with an outer wall thickness that permits a weight gain of about 15-17 mg being most preferable. As depicted in FIG. 3, for an 8 to 20 mg dose of israpidine provided in an oral osmotic delivery system, an outer wall thickness that permits a weight gain of about 18-26 mg is preferable, with an outer wall thickness that permits a weight gain of about 21-23 mg being most preferable. An approximate outer wall thickness in accordance with the present invention may be from about 45-75 μm, preferably from about 55-65 μm, with about 60 μm being most preferable.

In a further embodiment of the present invention, the outer coating may also comprise a flux-regulating agent to assist in regulating the fluid permeability through the outer coating. The flux-regulating agent can be a flux-enhancing agent or a flux-decreasing agent. Agents that produce a marked increase in permeability to fluid such as water are often essentially hydrophilic, while those that produce a marked decrease to fluids such as water are essentially hydrophobic. The flux regulators that may be used according to the present invention may include, e.g., polyhydric alcohols, polyalkylene glycols, polyalkylenediols and polyesters of alkylene glycols.

The oral osmotic delivery system of the present invention may optionally include one or more coatings in addition to the semi-permeable outer layer. Such additional coatings may be layers that are designed to modify the release characteristics of the dosage form, a coating to improve durability, shelf stability, ease of administration, or any other coatings typically used in conjunction with drug delivery systems. Enteric coatings are especially contemplated for use in accordance with the present invention, and may include, without limitation, cellulose acetate (including succinates and phthalates thereof), styrol maleic acid copolymers, hydroxypropyl methylcellulose phthalate, polyvinyl acetate phthalate, hydroxypropyl methylcellulose succinate acetate, hydroxyethyl cellulose phthalate, shellac, zein, acrylic resin, polymethacrylic acid/acrylic acid copolymer, and methacrylic acid copolymers. Where provided, these enteric coatings are applied to the oral osmotic drug delivery systems of the present invention using standard methods.

According to the present invention, the outer coating that substantially surrounds the drug layer and the osmotic layer comprises at least one opening. The opening allows the release of the drug from the delivery system and may include, for example, a passageway, an aperture, an orifice, or a bore. The opening in the outer coating may be formed by drilling, including mechanical and laser drilling. In some embodiments, the opening may be formed from a substance or polymer that erodes, dissolves, or is leached from the outer coating to leave an opening that allows the uniform release of the drug from the delivery system. A detailed description of osmotic passageways may be found, for example, in U.S. Pat. Nos. 3,845,770 and 3,916,899.

The outlet through the outer wall is preferably sized so as to permit the tablet to operate as an osmotic pump. Therefore, the outlet should not be so large as to permit a significant amount of the drug layer to diffuse outwardly through it relative to the amount of drug layer that is osmotically pumped out, and must not be so small as to cause pressure to accumulate within the tablet in excess of what would cause the outer wall to burst open. Accordingly, outlets in the range of about 4 microns to about 2000 microns in diameter will usually meet the above described functional criteria. Preferably, the outlet diameter is in the range of about 75 microns to about 350 microns.

6. Method of Preparing Dosage Form

The oral osmotic delivery device of the present invention may be manufactured using standard techniques.

By way of example, the drug and non-drug ingredients comprising the drug layer may be blended, optionally with a solvent, and mixed into a solid or semisolid form by conventional methods, e.g., ballmilling, calendering, stirring or rollmilling, to form granules having the desired % fines, where the granules are then pressed into a solid drug layer using a punch or die having the desired dimensions. Next, a layer of hydrophilic polymer is placed in contact with the drug layer. The layered tablet including a drug layer and an osmotic layer can then be fabricated by a conventional press-layering technique. Finally, the two-layer tablet is surrounded and coated with an outer wall. A passageway is laser drilled through the outer wall to contact the drug layer, thereby providing an oral osmotic delivery system.

It will be appreciated that alternative methods of forming the oral osmotic drug delivery system may also be used in accordance with the present invention.

7. Administration of Dosage Form

The present invention also provides methods of using an oral osmotic delivery system to provide controlled and sustained delivery of a drug. For example, the present invention provides a method of delivering a drug to a subject comprising administering to the subject an osmotic drug delivery system as described above.

After ingestion of the oral osmotic delivery system of the present invention, the osmotic activity gradient across the semi-permeable outer wall causes gastric fluid to be absorbed through the outer coating. The osmotic layer absorbs this fluid, and expands. The outer wall substantially retains its shape despite the expansion of the osmotic layer, thereby compressing the drug layer. The compression of the drug layer results in the release of the drug through the opening in the outer wall. As the drug is released, gastric fluid continues to be absorbed by the osmotic layer, which, in turn, causes release of more of the drug. Thus, the drug is released in a sustained and continuous manner over an extended time period. Preferably, the drug being administered is a dihydropyridine, such as israpidine, and the drug is released over a period of about 24 hours.

The oral osmotic drug delivery systems of the present invention result in an oral dosage form that provides a therapeutic effect for about 24 hours after administration, thereby permitting a more convenient once-a-day dosing schedule. Therapeutic levels of the drug are preferably achieved within about 1-3 hours after administration, and are more preferably achieved within about 2 hours after administration. The peak plasma level of the drug is preferably achieved from about 6-12 hours after administration, and is more preferably achieved within about 8-10 hours after administration. Greater than 50% of the peak plasma concentration is preferably maintained for about 12-24 hours, more preferably for about 17-20 hours.

The 16-hour cumulative dissolution (Q) of the oral osmotic drug delivery systems of the present invention is over 85%, preferably at least 90%, more preferably at least 92.5%, still more preferably at least 95%, and most preferably at least 97.5%. These Q values may be achieved in a variety of ways, as described herein. Notably, the Q values may be achieved by adjusting the amount of fines present in the drug layer, by adjusting the shape of the dosage form, or by adjusting the thickness of the outer coating, or by manipulating any combination of these variables.

According to a preferred embodiment in which the drug being administered is israpidine, the formulation results in a Cₘₐₓ of 2-5 ng/mL, more preferably 3-4 ng/mL. The AUC is preferably 50-80 ng·h/mL, more preferably 62-73 ng·h/mL.

Additional uses for the oral osmotic delivery system, and methods of administration the present invention are also envisioned. For example, and without limitation, the compositions of the present invention may also be useful in treating any conditions that may be alleviated by sustained release administration of therapeutic compositions. The oral osmotic delivery system may also be beneficially incorporated into preparations for use in the treatment of these and other conditions. The principles applied to the formation of the oral osmotic delivery system disclosed herein may also be applied to the formation of other delivery vehicles, such as sublingual, vaginal, rectal, etc.

### EXAMPLES

The following examples are merely illustrative of the present invention and should not be construed as limiting the scope of the invention in any way as many variations and equivalents that are encompassed by the present invention will become apparent to those skilled in the art upon reading the present disclosure.

1. Percent Fines

1.1 Preparation of Drug Layer Particles.

Polyethylene oxide, israpidine, hydroxypropyl methylcellulose, butylated hydroxytoluene, and alcohol (used as a solvent, later removed) were mixed using a Lodige mixer to produce granules for the drug layer of an oral osmotic tablet. These granules were formed in batches containing varying levels of fines. The batches of granules were then compressed to form drug layers for use in the cumulative dissolution and release rate testing described below. The drug layer was prepared by wet granulation, drying, milling, and blending operations.

1.2 Effect of % Fines on Cumulative Dissolution and % Release Rate.

The following examples show the effect of the particle size of the drug layer, defined as fines, on the 16-hour cumulative dissolution (Q) and the %/hr Release Rate (RR) of tablets containing 5 and 10 mg israpidine as the label claim amounts. In particular, the examples show that the Q value is directly proportional to the amount of fines in the drug layer. In addition, the examples show that the RR value is also directly proportional to the amount of fines in the drug layer. In each test, the following dissolution method was employed: USP dissolution test <711>, Apparatus 2, 50 rpm, 500 ml (for 5 mg tablet) or 1000 ml (for 10 mg tablets) of 0.2% N,N-dimethyl dodecylamine N-oxide (LDAO) or equivalent solvent, temperature 37° C.±0.5° C. Analytical methods for detecting isradipine amounts are preferably by HPLC using UV detection, but can be by any means known in the art. Preferably, Q is measured at the end of a 16 hr dissolution period, and RR is measured by a linear plot of the dissolution at 4, 8, and 12 hr time points.

Tables 1 and 2 show the dissolution profiles for 5 and 10 mg tablet batches as a function of % fines. The % dissolution at 4, 8 and 12-hour time points were determined and the Q value and RR value were calculated.

TABLE 1
Dissolution Data for 5 mg Tablets

| Batch Number | % Fines | Q | RR |
|---|---|---|---|
| 3041648AR | 16 | 89.7 | 8.3 |
| 3041648BR | 16 | 96.0 | 8.6 |
| 3041648CR | 16 | 91.4 | 8.6 |
| 3041648RD | 16 | 82.5 | 7.6 |
| 3041648RD | 16 | 76.1 | 7.1 |
| 3041648ER | 16 | 88.9 | 8.3 |
| 3042050AR | 18 | 97.2 | 8.8 |
| 3042050BR | 18 | 92.6 | 8.3 |
| 3042050RC | 18 | 88.6 | 7.7 |
| 3042050DR | 18 | 99.2 | 9.1 |
| 3042050REX | 18 | 93.5 | 8.6 |
| 3042051RA | 19 | 88.9 | 8.0 |
| 3042051BR | 19 | 95.0 | 8.5 |
| 3042051RC | 19 | 91.3 | 8.2 |
| 3042051DR | 19 | 86.8 | 8.0 |
| 3042051RE | 19 | 87.7 | 8.0 |
| 3044647A | 19 | 79.5 | 7.3 |
| 3044647B | 19 | 82.9 | 7.5 |
| 3044647C | 19 | 80.4 | 7.1 |
| 3044647D | 19 | 80.0 | 7.3 |
| 3044647E | 19 | 80.7 | 7.3 |
| 3044669A | 18 | 85.6 | 7.6 |
| 3044669B | 18 | 89.8 | 8.2 |
| 3044669C | 18 | 87.0 | 7.8 |
| 3044669D | 18 | 89.6 | 7.9 |
| 3044669E | 18 | 82.1 | 7.7 |
| 3044646B | 17 | 86.9 | 7.9 |
| 3044646C | 17 | 84.7 | 7.6 |
| 3044646D | 17 | 84.7 | 7.7 |
| 3044646E | 17 | 84.4 | 7.6 |
| 3045325A | 13.4 | 75.2 | 6.9 |
| 3045325B | 13.4 | 73.9 | 6.6 |
| 3045325D | 13.4 | 74.1 | 6.8 |
| 3045325E | 13.4 | 71.0 | 6.4 |
| 3045326A | 21.9 | 85.6 | 7.4 |
| 3045326B | 21.9 | 85.4 | 7.6 |
| 3045326E | 21.9 | 88.0 | 8.0 |
| 3045327A | 17.1 | 86.6 | 7.89 |
| 3045327B | 17.1 | 89.8 | 8.4 |
| 3045327C | 17.1 | 87.4 | 7.8 |
| 3045327D | 17.1 | 85.7 | 7.9 |
| 3045327E | 17.1 | 84.2 | 7.5 |
| 3045328A | 17.2 | 85.4 | 7.8 |
| 3045328B | 17.2 | 86.2 | 7.8 |
| 3045328C | 17.2 | 90.2 | 8.1 |
| 3045328D | 17.2 | 83.9 | 7.6 |
| 3045328E | 17.2 | 83.3 | 7.3 |
| 3045329A | 13.1 | 84.1 | 7.7 |
| 3045329B | 13.1 | 83.3 | 7.7 |
| 3045329C | 13.1 | 82.9 | 7.5 |
| 3045329D | 13.1 | 83.6 | 7.6 |
| 3045329E | 13.1 | 85 | 7.8 |
| 3045330A | 14.8 | 84.1 | 7.7 |
| 3045330B | 14.8 | 86.1 | 7.8 |
| 3045330C | 14.8 | 89.1 | 8 |
| 3045330D | 14.8 | 85.6 | 7.6 |
| 3045330E | 14.8 | 84.7 | 7.6 |
| 3047964RBX | 39.2 | 97.5 | 8.43 |
| 3047964RCX | 39.2 | 96.5 | 8.47 |
| 3047964RDX | 39.2 | 95.1 | 8.57 |
| 3047964REX | 39.2 | 92.9 | 8.34 |
| 3046533A | 35.9 | 98 | 8.88 |
| 3046533B | 35.9 | 96.3 | 8.95 |
| 3046533C | 35.9 | 95.9 | 8.99 |
| 3046533D | 35.9 | 96.7 | 8.79 |
| 3046533E | 35.9 | 95 | 8.81 |
| 3046534A | 37.3 | 98 | 8.88 |
| 3049171A | 28.7 | 92.6 | 8.18 |
| 3049171B | 28.7 | 93.3 | 8.11 |
| 3049171C | 28.7 | 91 | 8.12 |
| 3049171D | 28.7 | 89.1 | 7.69 |
| 3049171E | 28.7 | 89.8 | 7.93 |

TABLE 2
Dissolution Data for 10 mg Tablets

| Batch Number | % Fines | Q | RR |
|---|---|---|---|
| 3047431AR | 16.0 | 94.7 | 7.63 |
| 3047431RBX | 16.0 | 96.2 | 7.94 |
| 3047431CR | 16.0 | 92.8 | 7.39 |
| 3047431RDX | 16.0 | 96.9 | 8.13 |
| 3047431ER | 16.0 | 91.8 | 7.32 |
| 3047432RAX | 18 | 97.7 | 7.92 |
| 3047432BR | 18 | 97.1 | 8.04 |
| 3047432RCX | 18 | 99.7 | 8.3 |
| 3047432RDX | 18 | 98.5 | 8.21 |
| 3047432REX | 18 | 96.9 | 8 |
| 3047433RAX | 18 | 94.8 | 7.42 |
| 3047433RBX | 18 | 93.7 | 7.52 |
| 3047433RCX | 18 | 95.1 | 7.81 |
| 3047433DR | 18 | 94.8 | 7.71 |
| 3047433REX | 18 | 95.9 | 8.14 |
| 3048742A | 36.0 | 100.2 | 8.48 |
| 3048742B | 36.0 | 99.8 | 8.24 |
| 3048742C | 36.0 | 101.4 | 8.43 |
| 3048742D | 36.0 | 97.6 | 7.91 |
| 3048742E | 36.0 | 99.6 | 8.22 |
| 3048743A | 36.5 | 100.6 | 8.65 |
| 3048743B | 36.5 | 100.6 | 8.75 |
| 3048743C | 36.5 | 99.6 | 8.78 |
| 3048743D | 36.5 | 102 | 8.79 |
| 3048743E | 36.5 | 99.7 | 8.67 |
| 3048974A | 21.6 | 97.9 | 8.31 |
| 3048974B | 21.6 | 98.4 | 8.76 |
| 3048974C | 21.6 | 99.3 | 8.59 |
| 3048974D | 21.6 | 99.4 | 8.2 |
| 3048974E | 21.6 | 99.2 | 8.39 |
| 3049290A | 21.1 | 99.2 | 8.26 |
| 3049290B | 21.1 | 99.7 | 8.19 |
| 3049290C | 21.1 | 98.2 | 8.38 |
| 3049290D | 21.1 | 99.4 | 8.11 |
| 3049290E | 21.1 | 98.8 | 8.03 |
| 3049693XAX | 35.2 | 98.5 | 8.04 |
| 3049693XBX | 35.2 | 99.2 | 8.19 |
| 3049693XCX | 35.2 | 101.7 | 8.64 |
| 3049693XDX | 35.2 | 99.0 | 7.96 |
| 3049693XEX | 35.2 | 98.7 | 8.08 |

1.3 Relationship Between % Fines and 16-Hour Cumulative Dissolution.

Table 3 shows the relationship between % fines and 16-hour cumulative dissolution (Q) for 5 mg tablets manufactured with 36%, 46%, or 56% fines in the drug layer, and this relationship is also set forth in FIG. 2.

TABLE 3
Relationship between % Fines and 16-hour Cumulative Dissolution
Dissolution Data

| Description | % Fines | 16 h (Q) |
|---|---|---|
| 36% Fines | 36 | 87.5 |
| 46% Fines | 46 | 98.9 |
| 56% Fines | 56 | 105.3 |

1.4 Comparison of Dissolution Characteristics.

Table 4 provides a side-by-side comparison of the dissolution characteristics of 5 mg tablets produced using 39.2% fines and 21% fines in the drug layer over varying degrees of tablet gain (outer coating thickness).

TABLE 4
Dissolution Characteristics of Tablets Produced Using
39.2% Fines Vs. 21% Fines in the Drug Layer

| | Wt. | | | | |
|---|---|---|---|---|---|
| Time | Gain | Batch with 39.2% fines | | Batch with 21% fines | |
| (min) | (mg/tab) | Q | RR | Q | RR |
| 285 | 14.82 | 98.6 | 8.45 | 90.5 | 8.22 |
| 285 | 14.82 | 96.3 | 8.30 | 89.3 | 8.00 |
| 295 | 15.34 | 100.6 | 8.82 | 87.0 | 8.00 |
| 315 | 16.38 | 92.6 | 8.11 | 85.6 | 7.66 |
| 315 | 16.38 | 94.1 | 7.90 | 88.7 | 8.20 |
| 335 | 17.42 | 98.9 | 8.72 | 85.3 | 7.80 |
| 345 | 17.94 | 94.5 | 8.18 | 89.7 | 8.26 |
| 345 | 17.94 | 96.4 | 8.30 | 83.9 | 7.80 |

| Average | 96.2 | 8.33 | 87.1 | 7.96 |
|---|---|---|---|---|
| | Q | RR | | |
| Difference | 9.1 | 0.4 | | |

The results indicate that the increase in % fines in the drug layer corresponds to higher Q and higher RR values. In particular, the dissolution characteristics (Q and RR) of the tablets with 39.2% fines in the drug layer are significantly higher (t=6.9 and 2.4 for Q and RR, respectively) compared to batches with 21% fines in the drug layer.

2. Weight Gain

The relationship between tablet weight gain and 16-hour cumulative dissolution for a 5 mg tablet is set forth below in Table 5, and for a 10 mg tablet is set forth below in Table 6. The relationship between tablet weight gain and 16-hour cumulative dissolution (Q value) is also set forth in FIG. 3.

TABLE 5
Weight gain data for 5 mg Tablets

| Batch Number | Q | RR | wt. Gain (mg/tablet) |
|---|---|---|---|
| 3041648AR | 89.7 | 8.3 | 15.5 |
| 3041648BR | 96.0 | 8.6 | 16.1 |
| 3041648CR | 91.4 | 8.6 | 16.0 |
| 3041648RD | 82.5 | 7.6 | 15.8 |
| 3041648ER | 88.9 | 8.3 | 15.7 |
| 3042050AR | 97.2 | 8.8 | 15.7 |
| 3042050BR | 92.6 | 8.3 | 16.1 |
| 3042050DR | 99.2 | 9.1 | 15.7 |
| 3042051RA | 88.9 | 8.0 | 16.5 |
| 3042051BR | 95.0 | 8.5 | 16.2 |
| 3042051RC | 91.3 | 8.2 | 16.9 |
| 3042051DR | 86.8 | 8.0 | 16.3 |
| 3042051RE | 87.7 | 8.0 | 16.4 |
| 3044647A | 79.5 | 7.3 | 16.1 |
| 3044647B | 82.9 | 7.5 | 16.3 |
| 3044647C | 80.4 | 7.1 | 16.5 |
| 3044647D | 80.0 | 7.3 | 16.0 |
| 3044647E | 80.7 | 7.3 | 15.9 |
| 3044669A | 85.6 | 7.6 | 16.5 |
| 3044669B | 89.8 | 8.2 | 16.4 |
| 3044669C | 87.0 | 7.8 | 16.6 |
| 3044669D | 89.6 | 7.9 | 15.9 |
| 3044669E | 82.1 | 7.7 | 16.9 |
| 3044646B | 86.9 | 7.9 | 16.3 |
| 3044646C | 84.7 | 7.6 | 16.1 |
| 3044646D | 84.7 | 7.7 | 16.3 |
| 3044646E | 84.4 | 7.6 | 16.1 |
| 3045325A | 75.2 | 6.9 | 16.2 |
| 3045325B | 73.9 | 6.6 | 16.7 |
| 3045325D | 74.1 | 6.8 | 16.5 |
| 3045325E | 71.0 | 6.4 | 16.4 |
| 3045326A | 85.6 | 7.4 | 16.0 |
| 3045326B | 85.4 | 7.6 | 16.1 |
| 3045326E | 88.0 | 8.0 | 16.1 |
| 3045327A | 86.6 | 7.89 | 15.8 |
| 3045327B | 89.8 | 8.4 | 15.8 |
| 3045327C | 87.4 | 7.8 | 16.4 |
| 3045327D | 85.7 | 7.9 | 16 |
| 3045327E | 84.2 | 7.5 | 16 |
| 3045328A | 85.4 | 7.8 | 15.8 |
| 3045328B | 86.2 | 7.8 | 16.1 |
| 3045328C | 90.2 | 8.1 | 16.1 |
| 3045328D | 83.9 | 7.6 | 15.8 |
| 3045328E | 83.3 | 7.3 | 16.2 |
| 3045329A | 84.1 | 7.7 | 15.7 |
| 3045329B | 83.3 | 7.7 | 16.3 |
| 3045329C | 82.9 | 7.5 | 16 |
| 3045329D | 83.6 | 7.6 | 16.3 |
| 3045329E | 85 | 7.8 | 16.2 |
| 3045330C | 89.1 | 8 | 16.4 |
| 3045330D | 85.6 | 7.6 | 16.2 |
| 3045330E | 84.7 | 7.6 | 16.7 |
| 3047964RBX | 97.5 | 8.43 | 16.3 |
| 3047964RCX | 96.5 | 8.47 | 16.2 |
| 3047964RDX | 95.1 | 8.57 | 16.2 |
| 3047964REX | 92.9 | 8.34 | 16.7 |
| 3046533A | 98 | 8.88 | 14.7 |
| 3046533B | 96.3 | 8.95 | 14.8 |
| 3046533C | 95.9 | 8.99 | 16.1 |
| 3046533D | 96.7 | 8.79 | 15.6 |
| 3046533E | 95 | 8.81 | 15 |
| 3046534A | 98 | 8.88 | 14.7 |
| 3049171A | 92.6 | 8.18 | 16.2 |
| 3049171B | 93.3 | 8.11 | 16.2 |
| 3049171C | 91 | 8.12 | 16.2 |
| 3049171D | 89.1 | 7.69 | 15.9 |
| 3049171E | 89.8 | 7.93 | 15.8 |

TABLE 6
Weight gain data for 10 mg Tablets

| Batch Number | Q | RR | wt. Gain (mg/tablet) |
|---|---|---|---|
| 3047431AR | 94.7 | 7.63 | 22.21 |
| 3047431RBX | 96.2 | 7.94 | 22.26 |
| 3047431CR | 92.8 | 7.39 | 21.73 |
| 3047431RDX | 96.9 | 8.13 | 21.52 |
| 3047431ER | 91.8 | 7.32 | 22.01 |
| 3047432RAX | 97.7 | 7.92 | 20.87 |
| 3047432BR | 97.1 | 8.04 | 21.12 |
| 3047432RCX | 99.7 | 8.3 | 21.35 |
| 3047432RDX | 98.5 | 8.21 | 21.2 |
| 3047432REX | 96.9 | 8 | 20.88 |
| 3047433RAX | 94.8 | 7.42 | 21.48 |
| 3047433RBX | 93.7 | 7.52 | 21.7 |
| 3047433RCX | 95.1 | 7.81 | 21.5 |
| 3047433DR | 94.8 | 7.71 | 21.68 |
| 3047433REX | 95.9 | 8.14 | 21.39 |
| 3048742A | 100.2 | 8.48 | 20.8 |
| 3048742B | 99.8 | 8.24 | 20.93 |
| 3048742C | 101.4 | 8.43 | 21.02 |
| 3048742D | 97.6 | 7.91 | 21.65 |
| 3048742E | 99.6 | 8.22 | 21.7 |
| 3048743A | 100.6 | 8.65 | 20.78 |
| 3048743B | 100.6 | 8.75 | 20.54 |
| 3048743C | 99.6 | 8.78 | 21.35 |
| 3048743D | 102 | 8.79 | 21.75 |
| 3048743E | 99.7 | 8.67 | 21.55 |
| 3048974A | 97.9 | 8.31 | 21.02 |
| 3048974B | 98.4 | 8.76 | 21.61 |
| 3048974C | 99.3 | 8.59 | 20.83 |
| 3048974D | 99.4 | 8.2 | 19.98 |
| 3048974E | 99.2 | 8.39 | 19.84 |
| 3049290A | 99.2 | 8.26 | 21.91 |
| 3049290B | 99.7 | 8.19 | 22.18 |
| 3049290C | 98.2 | 8.38 | 21.08 |
| 3049290D | 99.4 | 8.11 | 21.78 |
| 3049290E | 98.8 | 8.03 | 20.98 |
| 3049693XAX | 98.5 | 8.04 | 20.5 |
| 3049693XBX | 99.2 | 8.19 | 21.4 |
| 3049693XCX | 101.7 | 8.64 | 21.3 |
| 3049693XDX | 99.0 | 7.96 | 21.6 |

3. Tablet Shape

The relationship between tablet shape and 16-hour cumulative dissolution (Q) is set forth below in Table 7. The increase in Q as the radius of curvature decreases was approximately 7% when the radius of curvature of the upper surface of a tablet containing a 5 mg dose of israpidine and having a tablet radius of 3.97 mm decreased from 9.55 mm to 5.93 mm. Further, despite the change in shape and increase in Q, the % release rate did not increase to the same degree (rising only up to 4.6% as the radius of curvature decreased).

TABLE 7
Relationship between Tablet Shape
and 16-hour Cumulative Dissolution

| | | | % Difference | % Difference |
|---|---|---|---|---|
| | Dissolution Data | | Q from “std” | RR from “std |
| Description | 16 h (Q) | % RR | % | % |
| Std Concave | 82.5 | 7.38 | 0.0 | 0.0 |
| (9.55 mm) | | | | |
| Deep Concave | 88.5 | 7.59 | 7.3 | 2.8 |
| (7.19 mm) | | | | |
| Extra Deep Concave | 88.2 | 7.72 | 6.9 | 4.6 |
| (5.93 mm) | | | | |

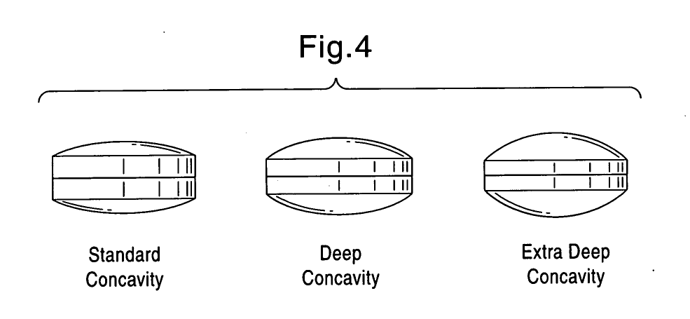

Examples of variations in tablet shape in accordance with the present invention are illustrated in FIG. 4.

It will, of course, be appreciated that the above description has been given by way of example only and that modifications in detail may be made within the scope of the present invention.

Throughout this application, various patents and publications have been cited. The disclosures of these patents and publications in their entireties are hereby incorporated by reference into this application, in order to more fully describe the state of the art to which this invention pertains.

The invention is capable of considerable modification, alteration, and equivalents in form and function, as will occur to those ordinarily skilled in the pertinent arts having the benefit of this disclosure.

While the present invention has been described for what are presently considered the preferred embodiments, the invention is not so limited. To the contrary, the invention is intended to cover various modifications and equivalent arrangements included within the spirit and scope of the detailed description provided above.

1-33. (canceled)

34. A method of making an oral osmotic drug delivery device comprising a drug layer comprising a drug and one or more non-drug ingredients, wherein the drug and non-drug ingredients are combined to form particles including an amount of fines; an osmotic layer; and a semi-permeable outer coating having a thickness value, wherein the outer coating substantially surrounds the drug layer and the osmotic layer, forms an upper surface having a radius of curvature, and defines at least one opening in the upper surface in communication with the drug layer, the method comprising controlling the amount of fines in the drug layer, the radius of curvature of the upper surface, and the thickness value of the outer coating to provide an oral osmotic drug delivery device having a 16-hour cumulative release of drug of at least 85% of a drug dose set forth in a label claim under the following conditions: USP dissolution test <711>, Apparatus 2, 50 rpm, 0.2% N,N-dimethyl dodecylamine N-oxide (LDAO) or equivalent solvent, temperature 37° C.±0.5° C.

35. The method of claim 34, wherein the 16-hour cumulative release of drug is at least 90% of a drug dose set forth in a label claim.

36. The method of claim 34, wherein the 16-hour cumulative release of drug is at least 92.5% of a drug dose set forth in a label claim.

37. The method of claim 34, wherein the 16-hour cumulative release of drug is at least 95% of a drug dose set forth in a label claim.

38. The method of claim 34, wherein the 16-hour cumulative release of drug is at least 97.5% of a drug dose set forth in a label claim.

39. The method of claim 34, wherein the drug comprises a dihydropyridine.

40. The method of claim 39, wherein the dihydropyridine is provided in a dose of from 2.5 to 20 mg.

41. The method of claim 34, wherein the drug comprises isradipine.

42. The method of claim 41, wherein the isradipine is provided in a dose of from 2.5 to 20 mg.

43. The method of claim 41, wherein the isradipine is provided in a dose of 5 mg.

44. The method of claim 41, wherein the isradipine is provided in a dose of 10 mg.

45. A method of delivering a drug to a subject in need thereof, comprising administering to the subject an oral osmotic tablet made by the method of claim 34.

46. The method of claim 45, wherein the drug comprises a dihydropyridine.

47. The method of claim 46, wherein the dihydropyridine is provided in a dose of from 2.5 to 20 mg.

48. The method of claim 45, wherein the drug comprises isradipine.

49. The method of claim 48, wherein the isradipine is provided in a dose of from 2.5 to 20 mg.

50. The method of claim 48, wherein the isradipine is provided in a dose of 5 mg.

51. The method of claim 48, wherein the isradipine is provided in a dose of 10 mg.

52. A method of once daily delivery of isradipine to a subject in need thereof, comprising administering once daily to the subject an oral osmotic drug delivery device comprising a drug layer comprising isradipine and one or more non-drug ingredients, wherein the isradipine and non-drug ingredients are combined to form particles including an amount of fines; an osmotic layer; and a semi-permeable outer coating having a thickness value, wherein the outer coating substantially surrounds the drug layer and the osmotic layer, forms an upper surface having a radius of curvature, and defines at least one opening in the upper surface in communication with the drug layer, wherein the oral osmotic delivery device is made by a method comprising controlling the amount of fines in the drug layer, the radius of curvature of the upper surface, and the thickness value of the outer coating to provide an oral osmotic drug delivery device having a 16-hour cumulative release of isradipine of at least 85% of an isradipine dose set forth in a label claim under the following conditions: USP dissolution test <711>, Apparatus 2, 50 rpm, 0.2% N,N-dimethyl dodecylamine N-oxide (LDAO) or equivalent solvent, temperature 37° C.±0.5° C.

53. The method of claim 52, wherein the administration provides a therapeutic effect for about 24 hours after administration.

54. The method of claim 52, wherein therapeutic levels of the drug isradipine are achieved within about 1-3 hours after administration.

55. The method of claim 52, wherein peak plasma levels of isradipine are achieved from about 6-12 hours after administration.

56. The method of claim 52, wherein greater than 50% of peak plasma levels are maintained for about 12-24 hours.

57. The method of claim 52, wherein the administration provides a Cₘₐₓ of 2-5 ng/mL.

58. The method of claim 52, wherein the administration provides an AUC of 50-80 ng·h/mL.
